SEQUENCE LISTING (25 sequences)

SEQ ID NO: 1 - DNA, 26 bp, Articifial Sequence
  Artificial Primer
gaaacggcctctgcggggagaagcaa

SEQ ID NO: 2 - DNA, 26 bp, Articifial Sequence
  Artificial Primer
gaaacggcctctgtggggagaagcaa

SEQ ID NO: 3 - DNA, 26 bp, Articifial Sequence
  Artificial Primer
gaaaccgcctctgcggggagaagcaa

SEQ ID NO: 4 - DNA, 17 bp, Articifial Sequence
  Artificial Primer
acccggcccgtccgtgg

SEQ ID NO: 5 - DNA, 17 bp, Articifial Sequence
  Artificial Primer
acctggcccgtccgtgg

SEQ ID NO: 6 - DNA, 20 bp, Articifial Sequence
  Artificial Primer
ccgggccaggttctcacacc

SEQ ID NO: 7 - DNA, 20 bp, Artificial Sequence
  Artificial Primer
cggggccaggttctcacacc

SEQ ID NO: 8 - DNA, 17 bp, Articifial Sequence
  Artificial Primer
tggcccctggtacccgt

SEQ ID NO: 9 - DNA, 17 bp, Articifial Sequence
  Artificial Primer
ggaggagcgaggggacc

SEQ ID NO: 10 - DNA, 20 bp, Articifial Sequence
  Artificial Primer
atctcggacccggagactcg

SEQ ID NO: 11 - DNA, 23 bp, Articifial Sequence
  Artificial Primer
cggatctcggacctagagactcg

SEQ ID NO: 12 - DNA, 25 bp, Artificial Sequence
  Artificial Primer
gtttacccggtttcattttcagttg

SEQ ID NO: 13 - DNA, 18 bp, Artificial Sequence
  Artificial Primer
ccatccccggcgacctat

SEQ ID NO: 14 - DNA, 18 bp, Artificial Sequence
  Artificial Primer
ccatcccgggcgatctat

SEQ ID NO: 15 - DNA, 21 bp, Artificial Sequence
  Artificial Primer
agcgaggggcccgcccggcga

SEQ ID NO: 16 - DNA, 21 bp, Artificial Sequence
  Artificial Primer
agcgaggtgcccgcccggcga

SEQ ID NO: 17 - DNA, 22 bp, Artificial Sequence
  Artificial Primer
gagatggggaaggctccccact

SEQ ID NO: 18 - DNA, 24 bp, Artificial Sequence
  Artificial Primer
gatccttcgtgtccccacagcacg

SEQ ID NO: 19 - DNA, 22 bp, Artificial Sequence
  Artificial Primer
gccgctgcactgtgaagctctc

SEQ ID NO: 20 - DNA, 22 bp, Artificial Sequence
  Artificial Primer
gccgctgcactgtgaagctctc

SEQ ID NO: 21 - DNA, 22 bp, Artificial Sequence
  Artificial Primer
gtttcttggagtactctacgtc

SEQ ID NO: 22 - DNA, 16 bp, Artificial Sequence
  Artificial Primer
gaggatttcgtgtacc

SEQ ID NO: 23 - DNA, 16 bp, Artificial Sequence
  Artificial Primer
gaggatttcgtgttcc

SEQ ID NO: 24 - DNA, 19 bp, Artificial Sequence
  Artificial Primer
ggtagttgtgtctgcacac

SEQ ID NO: 25 - DNA, 19 bp, Artificial Sequence
  Artificial Primer
ggtagttgtgtctgcatac

(19) United States
(12) Patent Application Publication
(10) Pub. No.: US 2005/0260662 A1
(43) Pub. Date: Nov. 24, 2005

(54) Genotyping of multiple loci with PCR for different loci amplification at different temperatures

(76) Inventor: Jiacheng Yang, Hillsboro, NJ (US)

Correspondence Address: Eric Mirabel;Bioarray Solutions, 35 Technology Drive, Warren, NJ, 07059, US

(21) Appl. No.: 11/131,632

(22) Filed: May 18, 2005

Related U.S. Application Data
(60) Provisional application No. 60/572,920, filed on May 20, 2004.

(51) Int. Cl. C12Q 1/68; C07H 21/04; C12P 19/34

(52) U.S. Cl. 435/6; 435/91.2; 536/24.3

(57) ABSTRACT

Disclosed is a method of performing simultaneous PCR amplification of several designated different loci in a sample each including a different target subsequence, using a set of pairs of forward and reverse primers, wherein the pairs are complementary to target subsequences, where different primer pairs are in different reaction chambers and the sample is also present in the reaction chambers, and wherein different primer pairs have different sequences. Different reaction chambers are provided different annealing temperatures, preferably at the same time, such that the annealing temperatures selected enhance annealing conditions for the primer pairs and the target subsequences within the reaction chambers. The method allows PCR to proceed more quickly, which is important to increase throughput in a multiplexed assay, and can be particularly important for HLA-typing in a transplantation setting.

### RELATED APPLICATIONS

This application claims priority to U.S. Provisional No. 60/572,920, filed May 20, 2004.

### BACKGROUND

Polymerase chain reaction (PCR) (as described, e.g., in U.S. Pat. No. 6,197,563, incorporated by reference) involves repetitive bi-directional DNA synthesis of a region of a nucleic acid, through extension of primers. PCR amplification of a DNA template requires two oligonucleotide primers, four deoxynucleotide triphosphates (dNTPs) with the appropriate base, magnesium ions, and a thermostable DNA polymerase. Three distinct events occur during each cycle of PCR reaction: (1) denaturation of the DNA template, (2) primer annealing, and (3) DNA synthesis by a thermostable polymerase. To achieve amplification of the region between the primers, these cycles are performed many times by cycling of the reaction temperature. When the reaction mixture is heated to 92-96° C., DNA denaturation occurs, resulting in generation of single-stranded DNA. After denaturation, temperature is adjusted to 37° C. to 65° C., at which temperature the oligonucleotide primers hybridize to their complementary single-stranded target sequences. The temperature selected at each step depends on factors including the homology of the primers for the target sequences, the length of the primers, as well as the base composition of the oligonucleotides. Extension of the oligonucleotide primer by a thermostable polymerase is usually carried out 68-72° C., depending on the optimum reaction temperature for the particular thermostable polymerase. The time required for copying the DNA template depends on the length of the PCR products as well as the DNA synthesis rate of the polymerase.

Several genetic loci in the human genome are associated with tissue-graft rejection. The loci that determine polymorphic cell surface glycoproteins that differ between individuals are designated the major histocompatibility complex (MHC). Two distinct classes of histocompatibility antigens have been characterized in humans: MHC Class I and MHC Class II. MHC Class I antigens are present on most types of mammalian cells, whereas MHC Class II antigens are restricted to a few types of cell, such as B lymphocytes, macrophages and dendritic cells. Unlike mice, human erythrocytes are devoid of Class I antigens, whereas they are ubiquitously expressed by human leukocytes. For this reason, the human MHC Class I antigens were called were called human leukocyte antigens (HLA). The HLA name is applied to both Class I and Class II antigens. The Class I molecules consist of a heavy chain (α-chain) and a common light chain (β₂-microglobulin). The heavy chains of the Class I molecules have six isoforms: HLA-A, -B, -C, -E, -F, and -G. In addition, there are HLA-H, -J, -K, and -L isoforms that are non-functional pseudogenes for the Class I molecules. The HLA Class II molecules are heterodimers composed of α and β chains roughly similar size. There are five isotypes of the Class II molecules: HLA-DM, -DO, -DP, -DQ and -DR. Genes encoding α chains of Class II molecules are designated as “A,” for example, as the “DRA gene.” The genes encoding β-chains are designated as “B,” for example, as the “DRB gene.” HLA-DR molecules have several functional β chain genes, as well as pseudogenes, and their number varies between chromosome 6. Different arrangements of β-chain genes are designated DRB haplotypes. Each of the haplotypes is associated with a characteristic antigen. For example, DR51, DR52, and DR53 antigens are products of the DRB5, DRB3 and DRB4 genes, respectively.

Differences in the Class I and Class II molecules expressed by transplant donors and recipients are the major stimuli of allograft rejection in clinical transplantation. These differences are due to extensive and complicated genetic polymorphism, that ensures different individuals inherit and express different combinations of Class I and II alleles. The protein encoded by an allele is called the haplotype. The combination of Class I and II allotypes expressed by an individual is the HLA type. The HLA type can be determined by using serological assays at the antigen level and by using DNA assays at the genetic level. Typing of HLA-A, B, C, DR and DQ loci are required for renal and bone marrow transplantation.

The genotype of the Class I and Class II MHC molecules can be determined by one of several methods, including sequence based typing, sequence specific primer (SSP) typing (also known as capture-mediated elongation detection, see, e.g., U.S. Pat. No. 6,307,039, incorporated by reference), sequence-specific oligonucleotide probe (SSOP) typing (also known as hybridization-mediated detection; see, e.g, U.S. Pat. No. 6,251,691, incorporated by reference), and reverse sequence-specific oligonucleotide probe (rSSOP) typing. PCR amplification of genomic DNA regions is required for all of these assays.

Different primers are required for amplifying different loci of the Class I and Class II molecules (as is the case when amplifying different loci of other genes). Because annealing temperatures for locus-specific PCR reactions are different, according to methods currently in use, PCR reactions for all the different loci, e.g., HLA-A, -B, -C, -DRB1, -DR52, -DQ, are not performed at the same time for the HLA DNA typing. Similarly, in other multiplexed genetic analysis (using hybridization or capture-mediated elongation assays) PCR reactions for all loci are not performed at the same time. Amplifying all loci simultaneously would be a way to significantly reduce the time required for PCR, and thereby reduce the time required for multi-loci and multiplexed genotyping analysis. Reducing the time required for PCR is important in applications such as organ donation, where a transplant cannot proceed from a cadaver until the genotyping is completed and a sufficiently close match in HLA type is confirmed. During a delay, the condition of either or both organ and intended recipient can deteriorate, which can determine the success of the transplant.

### SUMMARY

Disclosed is a method of performing simultaneous PCR amplification of several designated different loci in a sample each including a different target subsequence, using a set of pairs of forward and reverse primers, wherein the pairs are complementary to target subsequences, where different primer pairs are in different reaction chambers and the sample is also present in the reaction chambers, and wherein different primer pairs have different sequences. Different reaction chambers are provided different annealing temperatures, preferably at the same time, such that the annealing temperatures selected enhance annealing conditions for the primer pairs and the target subsequences within the reaction chambers. The temperatures are then further adjusted such that the following steps can proceed: primer annealing; primer elongation; elongation product de-annealing. The PCR amplification can be performed using a PTC-200 thermocycler from MJ Research.

The method allows PCR multi-loci amplification to proceed more quickly (when all reactions proceed simultaneously) than when the temperatures are sequentially changed and the reactions are run in sequence. This allows higher throughput for multiple samples and faster assays.

### BRIEF DESCRIPTION OF THE DRAWINGS

FIG. 1 illustrates the steps in the PCR amplification, followed by an assay, described and claimed herein.

FIG. 2 is a table showing the sequences of the forward and reverse primers used for amplification of various HLA loci.

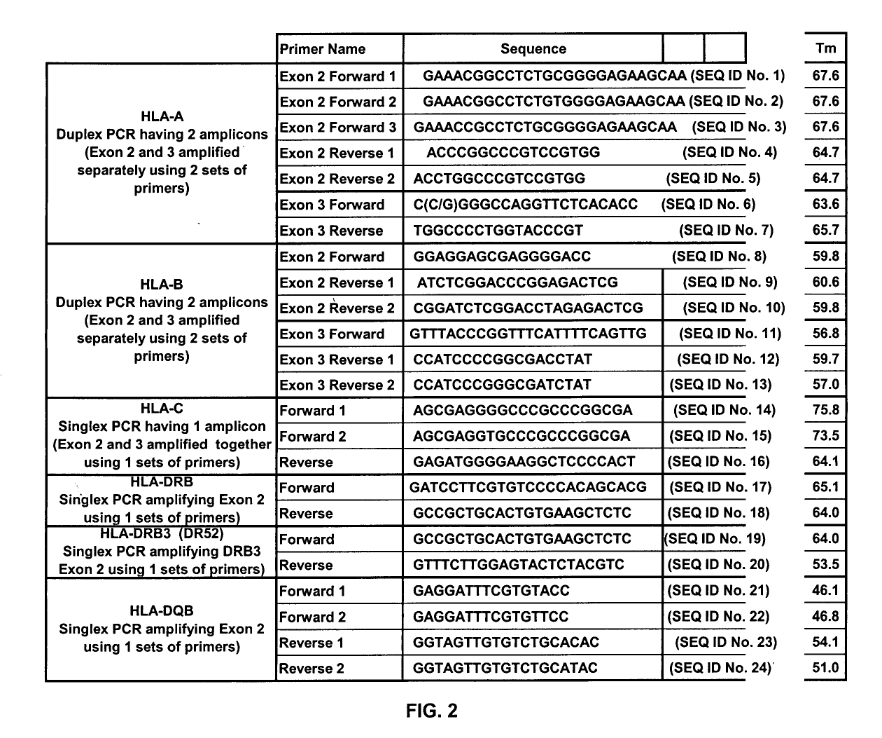

### DETAILED DESCRIPTION

The following examples aid in further understanding the invention claimed and described herein.

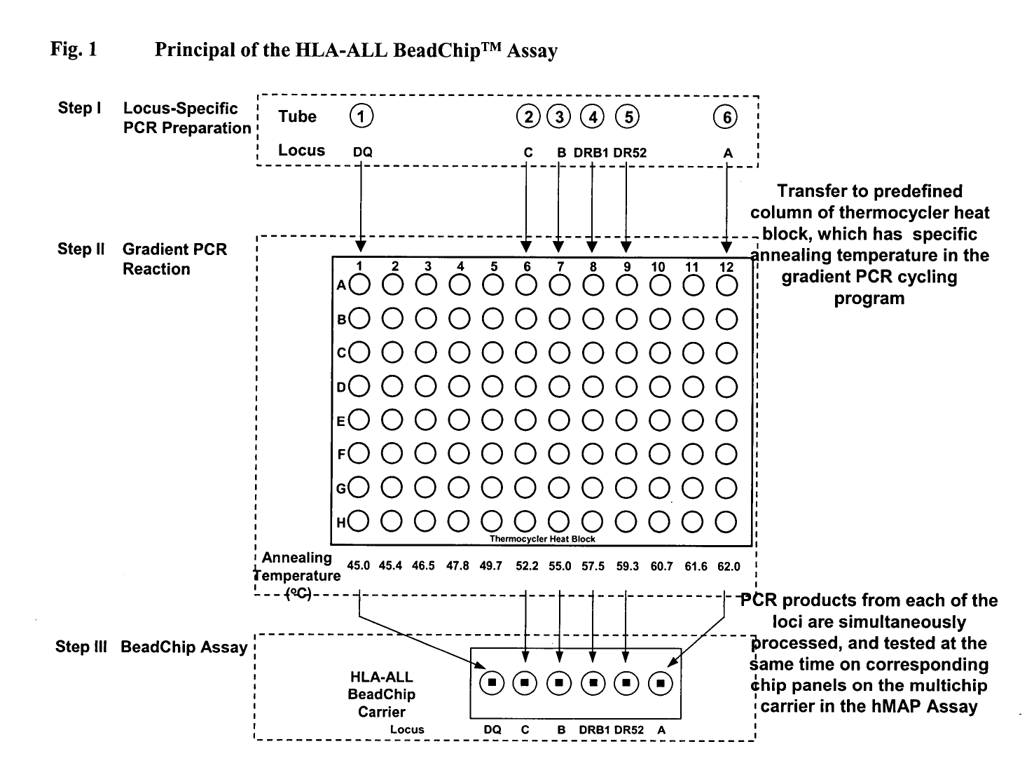

Locus-specific PCR amplification reactions can be prepared in individual test tubes according to methods known in the art. As illustrated in FIG. 1, each of the PCR reaction tubes includes a genomic DNA template, HLA locus-specific primers, dNTPs, reaction buffer, and thermostable polymerase. The dNTPs may be labeled, when the sample is to assayed using certain types of assays, particularly READ™ assays, as described in U.S. Pat. No. 6,514,771 and WO 01/98765.

Genomic DNA could be extracted from tissue and cells of a person, or a cadaver, according to methods known in the art. In addition, genomic DNA may be extracted from materials that contain blood, saliva and other body fluid samples, such as dried blood on filter paper. Methods for the extraction are known in the art. For example, the IsoCode filter paper card from Schleicher and Schull, Inc (Keene, N.H.) can be used for collection of blood sample. The dried blood on the IsoCode card can be used for DNA isolation according to manufacture's instruction. DNA isolated from the IsoCode card can be used as templates in PCR reactions for the HLA-All BeadChip assay.

A gradient PCR thermocycling program is set up in a gradient thermocycler, for example, the gradient thermocycler PTC-200 from MJ Research. Each PCR cycle has three steps: denaturation, annealing, and extension. In the annealing step, the temperature of the heat block is set to a gradient, according to the manufacturer's instruction. As shown in FIG. 1, the annealing temperature is gradually increased from 45° C. to 62° C. from column 1 to column 12, respectively. Temperature in other steps of the PCR (such as the temperature for denaturing or extension) is set to be the same in all 96 wells of the heat block. For example, a typical HLA-ALL gradient thermocycler program could be set ups as follows:

Section 1:

96° C. for 3 min, one cycle, then goes to Section 2.

(This section is required for activation of hot-start thermostable DNA polymerase)

Section 2:

96° C. for 20 seconds (DNA denaturation), then changes to 45° C. to 62° C. gradient temperature (annealing of primers to DNA templates) for 20 seconds (see FIG. 1A), then changes to 68° C. for 20 seconds (DNA synthesis by thermostable polymerase) Repeats Section 2 for 5 cycles, and then goes to Section 3.
   Section 3:
96° C. for 10 seconds (DNA denaturation), then changes to 45° C. to 62° C. gradient temperature (annealing of primers to DNA templates) for 15 seconds (see FIG. 1A), then changes to 68° C. for 20 seconds (DNA synthesis by thermostable polymerase) Repeats Section 2 for 30 cycles, and then goes to Section 4.
   Section 4:
68° C. for 10 min (for quenching the residual activity of the polymerase), then changes to 4° C. forever.

### EXAMPLE

Locus-specific PCR reactions prepared as described above are placed onto the heat block on a thermocycler in columns with predefined annealing temperatures. Annealing temperatures in specific column of the heat block match to the required annealing temperature, dependent on the length and nucleotide composition of the locus-specific primers.

DNA products amplified from PCR reactions could be analyzed by agarose gel (2%) electrophoresis, followed by ethedium bromide staining. The PCR products can be visualized with UV-translumination. As shown in FIG. 1, DNA products for HLA-DQ, -DR52, -C, -B, DRB1, and A loci are amplified simultaneously using purified genomic DNAs as templates in the PCR design. In addition, genomic DNAs isolated from dried blood on filter paper can be used as templates in the PCR amplification. PCR products from the dried blood templates are similar in quality to those from purified genomic DNAs. Next, PCR products from each of the loci are simultaneously fragmented by using hydrochloric acid followed by neutralization, using sodium hydroxide and heat-denaturation, in a well-known fragmentation protocol, previously described (see Wahl G M, Stem M, Stark G R. Efficient transfer of large DNA fragments from agarose gels to diazobenzyloxymethyl-paper and rapid hybridization by using dextran sulfate. Proc Nat'l Acad Sci USA 1979: 76:3683-7.). The processed samples could be used for on-chip hybridization assays and capture-mediated elongation assays (including the embodiments thereof as described in U.S. patent application Ser. Nos. 10/847,046 and 10/271,602, respectively).

The PCR is a rate-limiting step in the genotyping process, and by reducing the time that it takes to perform PCR using the temperature gradient PCR described herein, the speed of the genotyping process can be increased significantly.

Following PCR, one can react the amplicons with probes simultaneously, in different wells of a bead chip, also as shown in FIG. 1.

It should be understood that the embodiments, terms and expressions described herein are exemplary only, and not limiting, and that the scope of the invention is described only in the claims that follow, and includes all equivalents of the subject matter of those claims.

1. A method of performing simultaneous PCR amplification of several designated different loci in a sample each including a different target subsequence, using a set of pairs of forward and reverse primers, wherein the pairs are complementary to target subsequences, where different primer pairs are in different reaction chambers and the sample is also present in the reaction chambers, and wherein different primer pairs have different sequences, comprising:
providing different reaction chambers different annealing temperatures, such that the annealing temperatures selected enhance annealing conditions for the primer pairs and the target subsequences within the reaction chambers.

2. The method of claim 1 wherein the different annealing temperatures are provided to the different reaction chambers at the same time.

3. The method of claim 1 wherein the annealing temperatures are adjusted during the annealing process.

4. The method of claim 1 further including the step of adjusting the PCR reaction temperature such that, for the primer pairs, the following steps can proceed: primer annealing; primer elongation; elongation product de-annealing.

5. The method of claim 1 wherein the different primer pairs anneal to subsequences of the sample in an optimal manner, at different temperatures.

6. The method of claim 1 wherein different elongation products, which each incorporate a primer sequence, de-anneal from the sample at different temperatures.

7. The method of claim 1 wherein the PCR amplification is performed using a PTC-200 thermocycler from MJ Research.

8. An oligonucleotide having the sequence shown in any of SEQ ID Nos. 1-5 or 7-24, or an oligonucleotide with a complementary sequence.

9. An oligonucleotide having the sequence CCGGGCCAGGTTCTCACACC, or an oligonucleotide with a complementary sequence.

10. An oligonucleotide having the sequence CGGGGCCAGGTTCTCACACC, or an oligonucleotide with a complementary sequence.
